(19) United States
(12) Patent Application Publication
(10) Pub. No.: US 2002/0099186 A1
(43) Pub. Date: Jul. 25, 2002

(54) Processes for the preparation of alphaGal(1-3)betaGal(1-4)Glc-OR

(75) Inventors: Murray R. Ratcliffe, Cochrane (CA); Jonathan M. Gregson, Calgary (CA); Vivek P. Kamath, Calgary (CA); Robert E. Yeske, Calgary (CA)

Correspondence Address: BURNS DOANE SWECKER & MATHIS L L P, POST OFFICE BOX 1404, ALEXANDRIA, VA, 22313-1404, US

(73) Assignee: Synsorb Biotech, Inc., NW Calgary (CA)

(21) Appl. No.: 09/986,537

(22) Filed: Nov. 9, 2001

Related U.S. Application Data
(60) Provisional application No. 60/250,190, filed on Nov. 29, 2000.

(51) Int. Cl. C07H 15/04

(52) U.S. Cl. 536/18.6; 536/108

(57) ABSTRACT

Disclosed are novel synthetic processes for the preparation of the trisaccharide αGal(1→3)βGal(1→4)Glc-OR compounds.

### CROSS REFERENCE TO RELATED APPLICATIONS

This application claims priority to U.S. Provisional Patent Application Serial No. 60/250,190, filed Nov. 29, 2000 which application is incorporated herein in its entirety.

### BACKGROUND OF THE INVENTION

1. Field of the Invention

This invention is directed to novel synthetic processes for the preparation of the trisaccharide glycoside αGal(1→3)βGal(1→4)Glc-OR. Specifically, this invention is directed to a multi-step synthesis of this trisaccharide aglycon wherein the attachment of the aglycon is conducted after formation of the blocked βGal(1→4)Glc disaccharide.

2. References

The following publications, patents and patent applications are cited in this application as superscript numbers:

1 Heerze, et al., U.S. Pat. No. 5,484,773, for “Treatment of Antibiotic Associated Diarrhea”, issued Jan. 16, 1996.

2 Ratcliffe, et al., U.S. Pat. No. 5,079,353, for “Sialic Acid Glycosides, Antigens, Immunoadsorbents, and Methods for Their Preparation” issued Jan. 7, 1992.

3 Ekborg, et al., “Synthesis of Three Disaccharides for the Preparation of Immunogens Bearing Immunodeterminants Known to Occur on Glycoproteins”, Carbohydr. Res., 110:55-67 (1982).

4 Dahmen, et al., “2-Bromoethyl Glycosides:Applications in the Synthesis of Spacer-Arm Glycosides” Carbohydr. Res., 118:292-301. (1983).

5 Rana, et al., “Synthesis of Phenyl 2-Acetamido-2-Deoxy-3-O-α-L-Fucopyranosyl-β-D-Glucopyranoside and Related Compounds”, Carbohydr. Res., 91:149-157 (1981).

6 Amvam-Zollo, et al., “Type XIV Polysaccharide: Synthesis of a Repeating Branched Tetrasaccharide with Dioxa-Type Spacer-Arms”]Carbohydr. Res., 150:199-212 (1986).

7 Paulsen, et al., “Synthese Von Oligosaccharid-Determinanten Mit Amid-Spacer Vom Typ Des T-Antigens*”, Carbohydr. Res., 104:195-219 (1984).

8 Chernyak, et al., “New Type of Carbohydrate-Containing Synthetic Antigen: Synthesis of Carbohydrate-Containing Polyacrylamide Copolymers Having the Specificity of O:3 and O:4 Factors of Salmonella”, Carbohydr. Res., 128:269-282 (1984).

9 Fernadez-Santana, et al., “Glycosides of Monoallyl Diethylene Glycol. A new Type of Spacer Group for Synthetic Oligosaccharides”, J. Carbohydr. Chem., 8:531-537 (1989).

10 Lee, et al., “Synthesis of 3-(2-aminoethylthio)propyl Glycosides”, Carbohydr. Res., 37:193 et seq., (1974).

11 Lemieux, et al., “Properties of a “Synthetic” Antigen Related to the Human Blood-Group Lewis a”, J. Am. Chem. Soc., 97:4076-4083 (1975).

12 Pinto, et al., “Preparation of Glycoconjugates for Use as Artificial Antigens: A Simplified Procedure”, Carbohydr. Chem., 124:313-318 (1983).

13 Lubineau, et al., “Chemoenzyme synthesis of a disulfated Lewis pentasaccharide, a candidate ligand for human L-selectin”, Carbohydr. Res., 305: 501-509 (1998).

14 Szarek, et al., “Synthesis of 1,2,3-tri-O-β-lactosyl-D-threitol and 1-O-benzyl-2,3,4-tri-O-β-lactosyl-D-threitol”, Carbohdr. Res., 305: 27-31 (1998).

¹⁵ Kihlberg, et al., “Synthesis of a water-soluble serine-based neoglycolipid which can be covalently linked to a solid phase”, Carbohydr. Res., 258: 123-33 (1994).

¹⁶ Schmidt, et al., “Synthesis of β-lactosyl 1-thioceramide”, Leibigs Ann. Chem., 2: 185-7 (1991).

¹⁷ Szarek et al., “Synthesis and binding of D-galactose terminated ligands to human and rabbit asialoglycoprotein receptor,” Carbohydr. Res., 190: 203-18 (1989).

¹⁸ Takashi, et al., “Synthesis and activities of antitumor agents”, J. Med. Chem., 22: 247-50 (1979).

¹⁹ Shapiro, et al., “Synthetic studies on sphingolipids. Synthesis of cytolipin H and analogous lactosides”, Chem. Phys. Lipids, 1: 54-62 (1966).

²⁰ Ogawa, et al., Japanese Patent No. JP 63051396, for “Glycosphingolipids bearing a Lewis^{b} type antigenic determinant and a process for their preparation” issued Mar. 4, 1988.

²¹ Duthaler, et al, PCT Application No. WO 9847915, for “Preparation of neoglycoproteins as drugs”, issued Oct. 29, 1998.

²² Bundle, et al., “Stannic tetrachloride catalyzed glycosylation of 8-ethoxycarbonyloctanol by cellobiose, lactose and maltose octaacetates, synthesis of α and β-glycosidic linkages”, Can. J.

Chem., 57: 2085-90 (1979).

All of the above publications and patents are herein incorporated by reference in their entirety to the same extent as if each individual publication, patent or patent application was specifically and individually indicated to be incorporated by reference in its entirety.

State of the Art

Trisaccharide glycosides, such as the αGal(1→3)αGal(1→4)Glc-OR trisaccharide, have been disclosed by Heerze, et al.¹ as binding to toxin A and, accordingly, are useful in the treatment of antibiotic associated diarrhea mediated by, for example, Clostridium difficile.

Notwithstanding the beneficial properties of such trisaccharides, current synthetic processes for these compounds involve a multi-step process with overall low yields. This, in turn, has hampered the commercial development of these compounds.

Specifically, the complete chemical synthesis of oligosaccharide glycosides is a difficult task involving the generation of differentially protected or blocked hydroxyl groups on at least some of the hydroxyl groups of each of the saccharide units so as to provide a means to selectively remove one or more of the blocking groups thereby permitting the necessary reactions to be conducted on the unblocked hydroxyl group(s) as required to generate the desired compound.² Additionally, the numerous reaction procedures required in blocking and deblocking different hydroxyl groups necessitate a multi-step chemical synthetic procedure. It is desirable to maximize the generation of crystalline intermediates during the synthetic procedure to provide a facile means to purify the intermediates other than by chromatography or other equivalent means. In this regard, chromatography on intermediates and products achieved by large scale synthesis of trisaccharide glycosides is recognized as a time consuming process which can require the use of expensive equipment and is generally disadvantageous to an efficient large scale overall synthesis of the desired trisaccharide glycoside.

Contrarily, the use of glycosyltransferases to effect overall synthesis of the desired trisaccharide glycoside can be hindered by the lack of ready availability of the required glycosyltransferase, the difficulty in effecting large scale enzymatic reactions, the difficulty in coupling the desired saccharide to the nucleotide base required for coupling, etc.

### SUMMARY OF THE INVENTION

This invention is directed to novel processes for the overall chemical synthesis of αGal(1→3)βGal(1→4)Glc-OR which processes involve the derivation of a readily available lactose disaccharide derivative. Specifically and contrary to prior art processes, in one aspect, the processes of this invention defer attachment of the aglycon substituent (i.e., the R group) until after the lactose disaccharide structure has been fully protected. Surprisingly, by so deferring such an attachment, the overall yields of this trisaccharide are significantly improved.

In addition to the above, some of the intermediates generated by deferred attachment of this aglycon substituent are readily crystalline which further facilitates the overall synthetic process by eliminating chromatography steps which correspondingly facilitates the synthesis and can enhance the overall yield.

Additionally, in the herein described processes, the synthesis of the trisaccharide glycoside is completed in such a fashion that the number of manipulations at the disaccharide and trisaccharide levels is kept to a minimum and yield is improved. For example, notwithstanding the numerous reaction procedures involved in converting disaccharide 1 in FIG. 1 to αGal(1→3)βGal(1→4)Glc-O(CH₂)₈COOCH₃ (compound 11 in FIG. 2), the yield achieved by the processes of this invention for this conversion is approximately 24 percent.

Accordingly, in one of its process aspects, this invention is directed to a process for preparing αGal(1→3)βGal(1→4)Glc-OR compounds which process comprises:

(a) contacting β-R-lactoside represented by the formula:

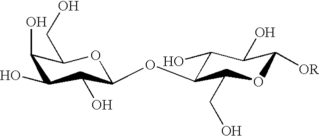

with at least a stoichiometric amount of a 2,2-dialkoxypropane under conditions to provide for β-R-3′,4′-dialkylacetal lactoside of the formula:

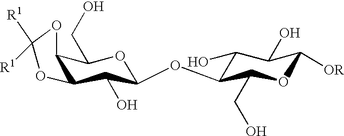

where R is an aglycon of at least 1 carbon atom and each R¹ is independently alkyl;

(b) acylating the compound produced in (a) above with at least 5 equivalents of an acyl halide under conditions to provide for β-R-3′,4′-dialkylacetal-2,2′,3,6,6′-penta-O-acyl-lactoside of the formula:

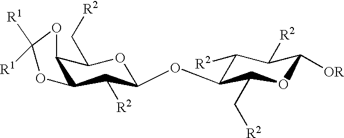

wherein R and R¹ are as defined above and R² is an acyl group;

(c) removing the dialkylacetal group from the compound produced in (b) above to provide for the β-R-2,2′,3,6,6′-penta-O-acyl-lactoside of the formula:

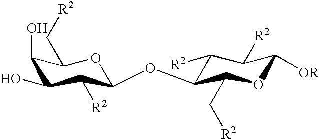

where R and R² are as defined above;

(d) acetylating the 4′-hydroxyl group of the the β-R-2,2′,3,6,6′-penta-O-acyl-lactoside by contacting the compound produced in (c) above with triethylorthoacetate under conditions to provide for the compound of the formula:

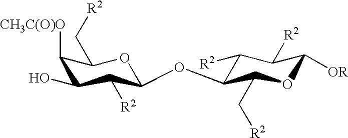

where R and R² are as defined above;

(e) contacting the compound produced in (d) above with β-thiobenzyl 2,3,4,6-tetra-O-benzyl-O-D-galactose under conditions to provide for a compound of the formula:

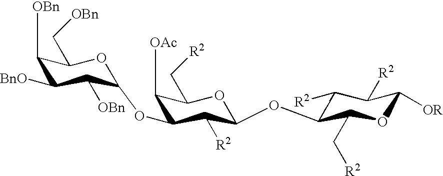

where R and R² are as defined above;

(f) removing each of the acetyl, benzyl and R² protecting groups in the compound produced in (e) above under conditions to provide for aglycon (α-D-galactopyranosyl)-(1→3)-O-(β-D-galactopyranosyl)-(1→4)-O-(β-D-glucopyranoside) which can also be referred to as αGal(1→3)βGal(1→4)Glc-OR and which is represented by the formula:

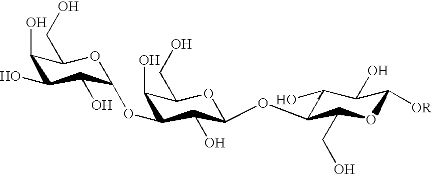

where R is as defined above.

Preferably, the β-R-lactoside represented by the formula:

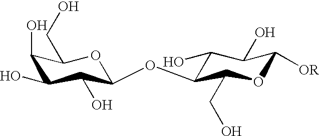

where R is as defined above, is prepared by the following process:

(i) contacting lactose of the formula:

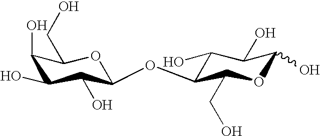

with at least 8 equivalents of benzoyl halide under conditions to provide for β-per-O-benzoyl-lactoside of the formula:

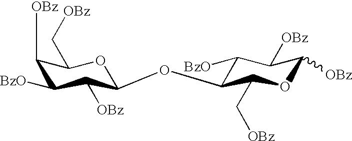

(ii) contacting the β-per-O-benzoyl-lactoside prepared in (i) above with at least a stoichiometric amount of hydrogen bromide under conditions to form the 1-α-bromo derivative of the formula:

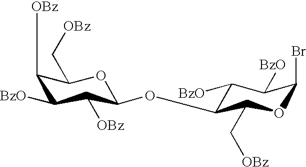

(iii) contacting the compound produced in (ii) above with a compound of the formula ROH under conditions to provide for β-R-2,2′,3,3′,4′,6,6′-hepta-O-benzoyl lactoside of the formula:

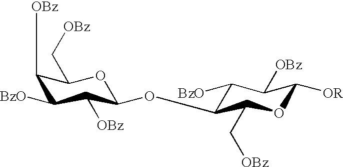

wherein R is an aglycon of at least 1 carbon atom;

(iv) contacting the compound produced in (iii) above under conditions to debenzoylate said compound to provide for β-R-lactoside

Preferably, the aglycon, R, contains from 1 to 20 carbon atoms and more perferably contains at least one functional group which allows attachment to a solid support. Most preferably, R is —(CH₂)₈COOCH₃.

Preferably, each R¹ is methyl.

Preferably, each R² is acetyl.

In another of its process aspects, this invention is directed to a process for the synthesis of αGal(1→3)βGal(1→4)Glc-O(CH₂)₈—COOCH₃ which process comprises:

(a) contacting lactose of the formula:

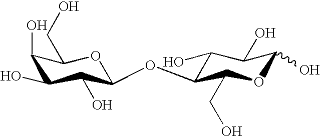

with at least 8 equivalents of benzoyl halide under conditions to provide for β-per-O-benzoyl-lactoside of the formula:

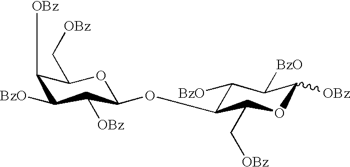

(b) contacting the β-per-O-benzoyl-lactoside prepared in (a) above with at least a stoichiometric amount of hydrogen bromide under conditions to form the 1-α-bromo derivative of the formula:

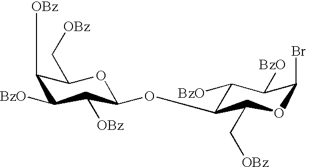

(c) contacting the compound produced in (b) above with at least a stoichiometeric amount of a compound of the formula ROH under conditions to provide for β-OR-2,2′,3,3′,4′,6,6′-hepta-O-benzoyl lactoside of the formula:

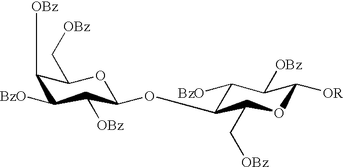

wherein R is —(CH₂)₈COOCH₃;

(d) contacting the compound produced in (c) above under conditions to debenzoylate said compound to provide for β-R-lactoside of the formula:

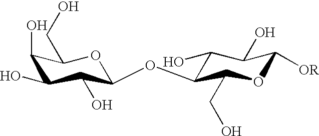

wherein R is as defined above;

(e) contacting the β-OR-lactoside produced in (d) above with at least a stoichiometric amount of a 2,2-dialkoxypropane under conditions to provide for β-R-3′,4′-dialkylacetal lactoside of the formula:

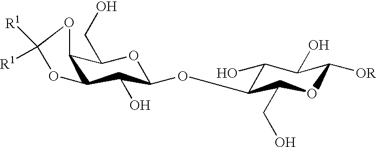

where each R¹ is independently alkyl;

(f) acylating the compound produced in (e) above with at least 5 equivalents of an acyl halide under conditions to provide for β-R 3′,4′-dialkylacetal-2,2′,3,6,6′-penta-O-acyl-lactoside of the formula:

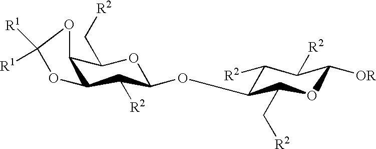

wherein R and R¹ are as defined above and R² is an acyl group;

(g) removing the dialkylacetal group from the compound produced (f) above to provide for the β-R-2,2′,3,6,6′-penta-O-acyl-lactoside of the formula:

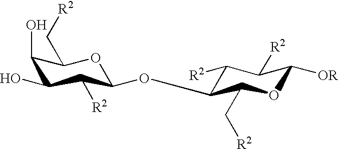

where R and R² are as defined above;

(h) acetylating the 4′-hydroxyl group of the the β-R-2,2′,3,6,6′-penta-O-acyl-lactoside by contacting the compound produced in (g) above with triethylorthoacetate under conditions to provide for the compound of the formula:

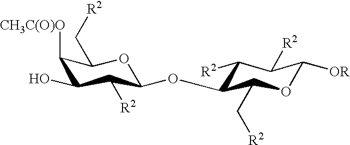

where R and R² are as defined above;

(i) contacting the compound produced in (h) above with β-thiobenzyl 2,3,4,6-tetra-O-benzyl-O-D-galactose under conditions to provide for a compound of the formula:

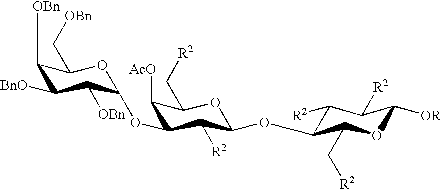

where R and R² are as defined above;

(j) removing each of the acetyl, benzyl and R² protecting groups in the compound produced in (i) above under conditions to provide for αGal(1→3)βGal(1→4)Glc-OR and which is represented by the formula:

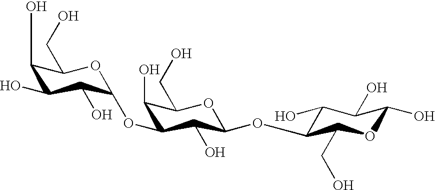

where R is as defined above.

### BRIEF DESCRIPTION OF THE DRAWINGS

FIGS. 1-2 illustrate the preferred synthetic procedure for the preparation of 8-methoxycarbonyloctyl (α-D-galactopyranosyl)-(1→3)-O-(β-D-galactopyranosyl)-(1→4)-O-(β-D-glucopyranoside) starting with lactose.

### DETAILED DESCRIPTION OF THE INVENTION

This invention is directed to processes for the preparation of the trisaccharide αGal(1→3)βGal(1→4)Glc-OR.

Prior to discussing this invention in further detail, the following terms will first be defined:

The term “lactose” refers to the disaccharide βGal(1→4)Glc which can be represented by the formula:

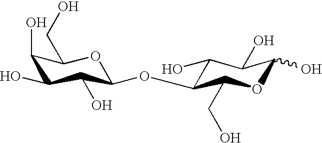

The term “lactoside” refers to the disaccharide βGal(1→4)Glc-OR where R is an aglycon of at least one carbon atom.

The term “aglycon of at least one carbon atom” refers to non-saccharide containing residues having at least one carbon atom, preferably from 1 to 20 carbon atoms and more preferably from 1 to 10 carbon atoms. Even more preferably, the aglycon is selected from the group consisting of —(A)—Z wherein A represents a bond, an alkylene group of from 2 to 10 carbon atoms, and a moiety of the form —(WG)ₙ— wherein n is an integer equal to 1 to 5; W is a straight or branched chain alkylene group of from 2 to 10 carbon atoms optionally substituted with 1 to 3 substituents selected from the group consisting of aryl of 6 to 10 carbon atoms and aryl of from 6 to 10 carbon atoms substituted with from 1 to 3 substituents selected from the group consisting of amino, hydroxyl, halo, alkyl of from 1 to 4 carbon atoms and alkoxy of from 1 to 4 carbon atoms; G is selected from the group consisting of a bond, O, S and NH; and Z is selected from the group consisting of hydrogen, methyl, phenyl, nitrophenyl and, when G is not oxygen, sulphur or nitrogen and when A is not a bond, then Z is also selected from the group consisting of —OH, —SH, —NH₂, —NHR³, —N(R³)₂, —C(O)OH, —C(O)OR³, —C(O)NH—NH₂, —C(O)NH₂, —C(O)NHR³, —C(O)N(R³)₂, and —OR⁴ wherein each R³ is independently alkyl of from 1 to 4 carbon atoms and R⁴ is an alkenyl group of from 3 to 10 carbon atoms.

Preferably, the aglycon contains a functional group or can be derivatized to contain a functional group which allows the aglycon to covalently bond to a solid support thereby providing for a compatible linker arm between the oligosaccharide and the solid support. Such functional groups are well known in the art and are selected to bind to a complementary functional group on the solid support to form a covalent bond or linkage. Such complementary functional groups include a first reactive group present on either the aglycon or the solid support and a second reactive group found on either the solid support or the aglycon and include, by way of example, those set forth below:

| First Reactive Group | Second Reactive Group | Linkage |
|---|---|---|
| hydroxyl | isocyanate | urethane |
| amine | epoxide | β-hydroxyamine |
| sulfonyl halide | amine | sulfonamide |
| carboxyl acid | amine | amide |
| hydroxyl | alkyl/aryl halide | ether |
| aldehyde | amine/NaCNBH₄ | amine |
| ketone | amine/NaCNBH₄ | amine |
| amine | isocyanate | urea |

Numerous aglycons are known in the art. For example, an aglycon comprising a p-nitrophenyl group (i.e., —OR═—OC₆H₄(p)NO₂) has been disclosed by Ekborg, et al.³ At the appropriate time during synthesis, the nitro group is reduced to an amino group which can be protected as N-trifluoroacetamido. When desired, the trifluoroacetamido group is removed thereby unmasking the amino group which can be used for coupling to a solid support.

An aglycon containing sulfur is disclosed by Dahmen, et al.⁴ Specifically, the aglycon is derived from a 2-bromoethyl group which, in a substitution reaction with thionucleophiles, has been shown to lead to aglycons possessing a variety of terminal functional groups such as —OCH₂CH₂SCH₂CO₂CH₃ and —OCH₂CH₂SC₆H₄-p—NH₂ both of which can be used to couple this aglycon to a solid support.

Rana, et al.⁵ discloses a 6-trifluoroacetamidohexyl aglycon [—O(CH₂)₆NHCOCF₃] in which the trifluoroacetamido protecting group can be removed unmasking the primary amino group which, again, can be used to couple this aglycon to a solid support.

Other exemplifications of known aglycons include the 7-methoxycarbonyl-3,6-dioxaheptyl aglycon⁶ (—OCH₂CH₂)₂OCH₂CO₂CH₃; the 2-(4-methoxycarbonylbutancarboxamido)ethyl⁷ [—OCH₂CH₂NHC(O)(CH₂)₄CO₂CH₃]; and the allyl aglycon⁸ (—OCH₂CH═CH₂) which, by radical co-polymerization with an appropriate monomer, leads to co-polymers. Other allyl linking aglycons⁹ are known [e.g., —O(CH₂CH₂O)₂CH₂CH═CH₂]. Additionally, allyl linking arms can be derivatized in the presence of 2-aminoethanethiol¹⁰ to provide for the aglycon of the formula —OCH₂CH₂CH₂SCH₂CH₂NH₂. As before, such aglycons permit covalent linkage to a solid support containing reactive groups complementary to the groups on the aglycon. That is to say that a complementary reactive group on the solid support is one which will selectively react with a reactive functionality on the aglycon to provide for covalent linkage therebetween.

Additionally, as shown by Ratcliffe, et al.², the aglycon R¹ group can be an additional saccharide-OR⁴ or an oligosaccharide-OR⁴ at the reducing sugar terminus (where R⁴ is an aglycon as defined above).

Preferably, the aglycon moiety is a —(CH₂)₈COOCH₃.

The term “compatible linker arm” refers to a moiety which serves to space the oligosaccharide structure from the solid support and which is bifunctional wherein one functional group is capable of binding to a reciprocal functional group of the support and the other functional group is capable of binding to a reciprocal functional group of the oligosaccharide structure. Compatible linker arms preferred in the present invention are non-peptidyl spacer arms.

In a preferred embodiment, the aglycon attached to the oligosaccharide comprises functionality or can be derivatized to contain functionality which permits attachment of the aglycon to the solid support. For example, allyl groups, nitro groups, carboxyl esters can be derivatized via conventional synthetic methods to permit covalent linkage to a compatible functional group on the surface of a solid support. Epoxides, amines, hydrazines, and similar groups on the aglycon can be reacted directly with a compatible functional group on the surface of a solid support to effect covalent linkage.

The term “solid support” refers to an inert, solid material to which the oligosaccharide sequences may be bound via a compatible linker arm. Where use is in vivo, the solid support will be biocompatible and preferably non-immunogenic.

For purpose of this application, all sugars are referenced using conventional three letter nomenclature. All sugars are assumed to be in the D-form unless otherwise noted, except for fructose, which is in the L-form. Further all sugars are in the pyranose form.

The term “pharmaceutically acceptable salts” include any and all pharmaceutically acceptable addition salts of αGal(1→3)βGal(1→4)Glc-OR compounds derived from a variety of organic and inorganic counter ions well known in the art and include, by way of example only, sodium, potassium, calcium, magnesium, ammonium, tetraalkylarnmonium, and the like.

The term “removable blocking group” refers to any group which when bound to one or more hydroxyl groups of either or both galactose units or the glucose unit, used to prepare the αGal(1→3)βGal(1→4)Glc-OR compounds described herein, prevents reactions from occurring at these hydroxyl groups and which protecting groups can be removed by conventional chemical and/or enzymatic procedures to reestablish the hydroxyl group without otherwise unintentionally altering the structure of the compound. The particular removable blocking group employed is not critical and preferred removable hydroxyl blocking groups include conventional substituents such as benzyl, benzoyl, acetyl, chloroacetyl, benzylidene, t-butylbiphenylsilyl and any other group that can be introduced onto a hydroxyl functionality and later selectively removed by conventional methods in mild conditions compatible with the nature of the product.

As used herein, “alkyl” refers to alkyl groups preferably having from 1 to 6 carbon atoms and more preferably 1 to 4 carbon atoms. This term is exemplified by groups such as methyl, ethyl, n-propyl, iso-propyl, n-butyl, tert-butyl, sec-butyl, n-pentyl, n-hexyl, 1,2-dimethylbutyl, and the like.

“Aryl” refers to an unsaturated aromatic carbocyclic group of from 6 to 10 carbon atoms having a single ring (e.g., phenyl) or multiple condensed rings (e.g., naphthyl).

“Alkaryl” refers to alkyl groups containing 1 to 2 aryl substituents thereon. Such groups preferably comprise from 7 to 18 carbon atoms and are represented by benzyl, —CH₂CH₂-φ, —CH(φ)₂ and the like.

“Acyl” refers to the group R⁵C(O)O— where R⁵ is alkyl, aryl, alkaryl and the like. In the processes of this invention, the R⁵ substituent of the acyl group is preferably benzyl.

“Acyl halide” refers to the group R⁵C(O)X where R⁵ is as defined above and X is a halide selected from the group consisting of chloro and bromo.

Methodology

The αGal(1→3)βGal(1→4)Glc-OR compounds described herein may be prepared by the following general methods and procedures. It should be appreciated that where typical or preferred process conditions (e.g., reaction temperatures, times, mole ratios of reactants, solvents, pressures, etc.) are given, other process conditions may also be used unless otherwise stated. Optimum reaction conditions may vary with the particular reactants or solvents used, but such conditions can be determined by one skilled in the art by routine optimization procedures.

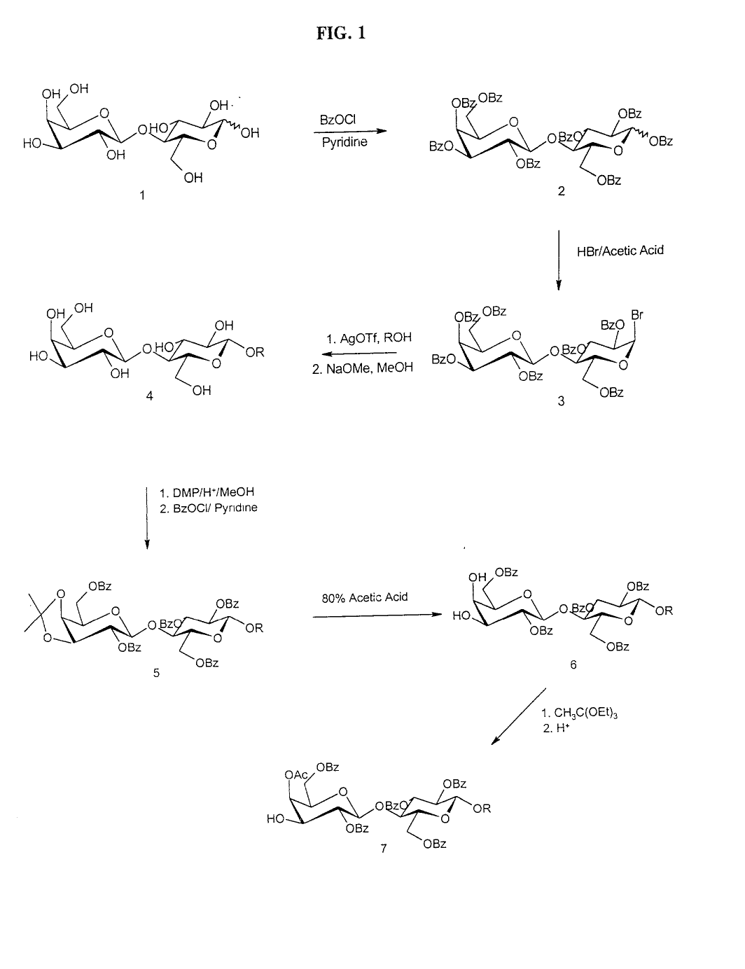

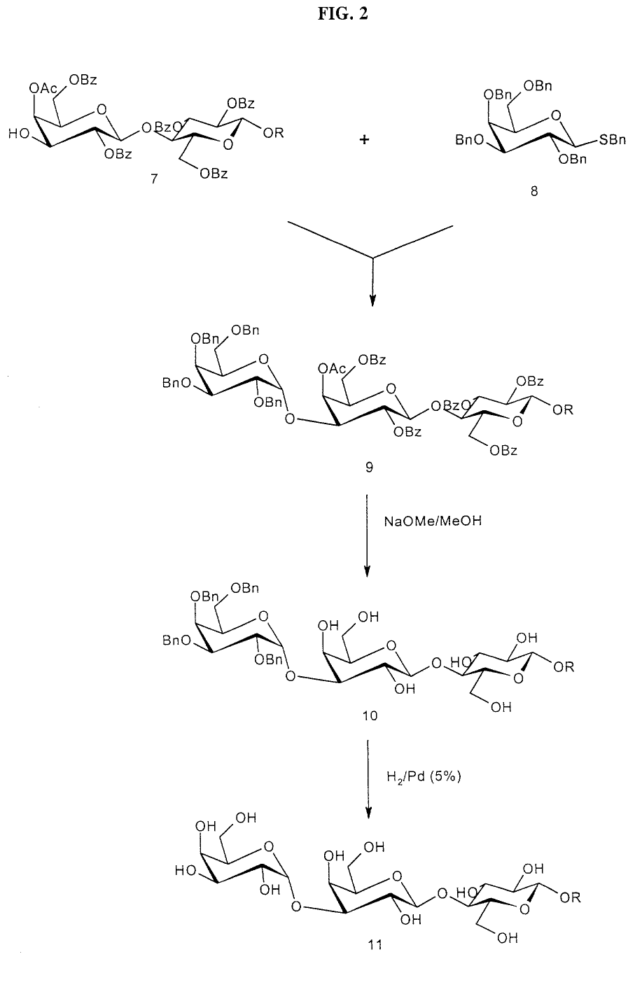

FIGS. 1 and 2 illustrate preferred reaction schemes using a —(CH₂)₈COOCH₃ aglycon. It is understood that other aglycons could be employed in the reactions below merely by replacing HO(CH₂)₈COOCH₃ with other alcohols of the formula HOR where R is as defined herein.

Generally, the synthetic processes of this invention start with the disaccharide lactose. This compound is fully hydroxyl protected by reaction with an excess of benzoyl chloride in an inert diluent as shown in reaction

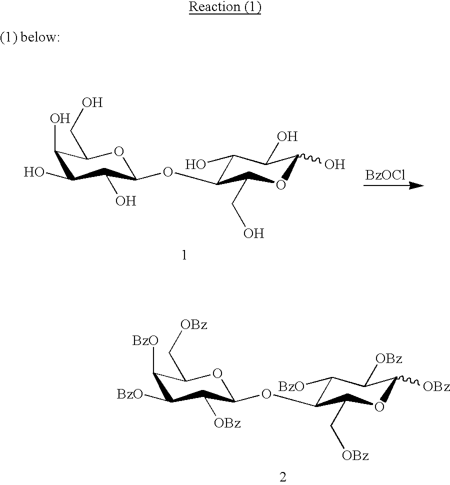

Reaction (1) is preferably conducted by combining lactose, compound 1, with an excess, i.e., at least 8 stoichiometric equivalents, of benzoyl chloride in a suitable inert diluent. Preferably, the amount of benzoyl chloride employed in the reaction is from at least 8 to about 16 equivalents and more preferably about 12 equivalents.

In one embodiment, an organic base such as triethylamine, diisopropylethylamine and the like is added to the reaction mixture to scavenge the acid generated. In another embodiment, an diluent containing a basic group is employed. In this case, pyridine is employed as the preferred diluent.

The reaction is conducted under conditions to provide for per-O-benzoyl-β-D-lactoside, compound 2. Preferably, the reaction is conducted at a temperature of from about 20° to about 80° C. and more preferably at about 65° C. for a period of from about 6 to about 48 hours. Specifically, it has been found that maintaining the reaction at about 65° C. significantly reduces reaction time as compared to reaction at room temperature. After reaction completion, methanol is preferably added to the reaction mixture to destroy any unreacted benozyl chloride. The resulting product is recovered by conventional methods including stripping of the solvent, chromatography, crystallization and the like, or can be used directly in the next reaction procedure without purification and/or isolation.

In one preferred embodiment, the reaction mixture is concentrated, dissolved in methylene chloride, washed with water, an acidic aqueous solution and a basic solution. The resulting product is then dried over sodium sulfate, filtered and is used directly in reaction (2) as described below.

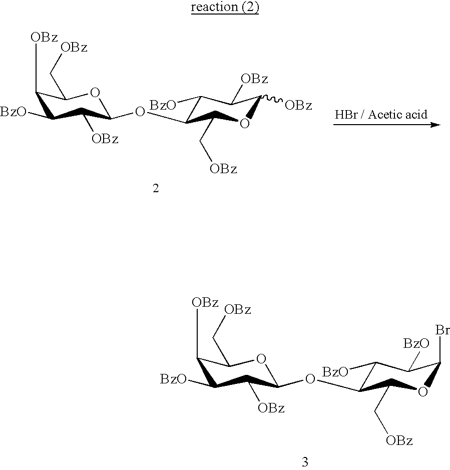

Reaction (2) is preferably conducted by combining per-O-benzoyl-β-D-lactoside, compound 2, with an excess of hydrogen bromide/acetic acid in a suitable inert diluent. Preferably, the amount of hydrogen bromide employed in the reaction is from at least 1 to about 10 equivalents and more preferably about 4 equivalents.

The reaction is conducted under conditions to provide for 2,3,6,2′,3′,4′,6′-hepta-O-benzoyl-α-D-lactosyl bromide, compound 3. Preferably, the reaction is conducted at a temperature of from about 10° to about 40° C., more preferably from about 10° C. to about 30° C. and still more preferably at about room temperature for a period of from about 6 to about 32 hours. After reaction completion, the resulting product is recovered by conventional methods including stripping of the solvent, chromatography, crystallization and the like, or can be used directly in the next reaction procedure without purification and/or isolation.

In one preferred embodiment, the reaction solution is washed repeatedly with a basic aqueous solution until the pH of the aqueous wash solution is about 7. The organic solution is then concentrated and recovered by conventional methods to provide for compound 3.

2,3,6,2′,3′,4′,6′-Hepta-O-benzoyl-α-D-lactosyl bromide, compound 3, is converted to the 8-methoxycarbonyloctyl-2,3,6,2′,3′,4′,6′-hepta-O-benzoyl-β-D-lactoside, compound 3a, as shown in reaction (3) below:

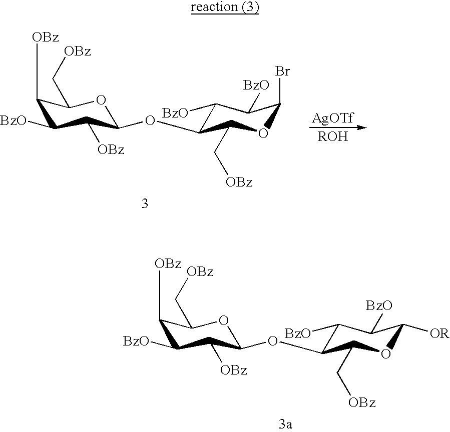

where R is —(CH₂)₈COOCH₃.

Reaction (3) involves the formation of a glycosidic linkage on the anomeric carbon atom of the reducing sugar wherein the benzoyl protected lactosyl bromide, 3, is reacted under catalytic conditions well known in the art with an aglycon which possesses one free hydroxyl at the position where the glycosidic linkage is to be established. A large variety of aglycon moieties are known in the art and can be attached with the proper configuration to the anomeric center of the reducing unit.

Reaction (3) is preferably conducted by combining 2,3,6,2′,3′,4′,6′-hepta-O-benzoyl-α-D-lactosyl bromide, compound 3, with an excess of the alcohol ROH (i.e., HO—(CH₂)₈COOCH₃) in a suitable inert diluent. Suitable diluents include, by way of example, chloroform, methylene chloride, and the like. Preferably, the amount of the alcohol employed in the reaction is from at least 1 to about 1.5 equivalents and more preferably about 1.2 equivalents.

The reaction is conducted under conditions to provide for 8-methoxycarbonyloctyl-2,3,6,2′,3′,4′,6′-hepta-O-benzoyl-β-D-lactoside, compound 3a. Preferably, the reaction is conducted at a temperature of from about 10° to about 50° C. and more preferably at about room temperature for a period of from about 0.1 to about 10 hours. A stoichiometric excess and preferably about 1.3 equivalents of silver trifluoromethane sulfonate (AgOTf) is employed in the reaction to assist in conversion of the lactosyl bromide to compound 4. In addition, a stoichiometric excess and preferably about 1.1 equivalents of 1,1′,3,3′-tetramethyl urea is employed in the reaction to enhance the overall yield of the desired β-D-lactoside.

After reaction completion, the reaction solution is preferably neutralized with the addition of a trialkyl amine such as triethyl amine, diisopropylethyl amine and the like to provide for compound 4. The resulting product is then recovered by conventional methods including stripping of the solvent, chromatography, crystallization and the like, or can be used directly in the next reaction procedure without purification and/or isolation.

The benzoyl blocking groups on the 8-methoxycarbonyloctyl-2,3,6,2′,3′,4′,6′-hepta-O-benzoyl-β-D-lactoside, compound 3a, are then removed by conventional methods as shown in reaction (4) below to provide for the desired 8-methoxycarbonyloctyl-β-D-lactoside, compound 4.

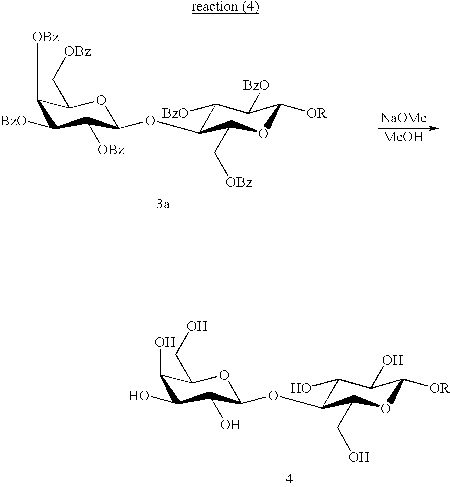

The reaction is conducted under conditions to provide for 8-methoxycarbonyloctyl-β-D-lactoside, compound 4. Preferably, the reaction is conducted using an excess of sodium methoxide in methanol at a temperature of from about 30° to about 60° C. and more preferably at about 45° C. The reaction is neutralized by addition of acetic acid. The resulting product is then recovered by conventional methods including stripping of the solvent, crystallization and the like.

8-Methoxycarbonyloctyl-β-D-lactoside, compound 4, is then converted to 8-methoxycarbonyloctyl-3′,4′-O-isopropyidene-β-D-lactoside, compound 4a, as shown in reaction (5) below:

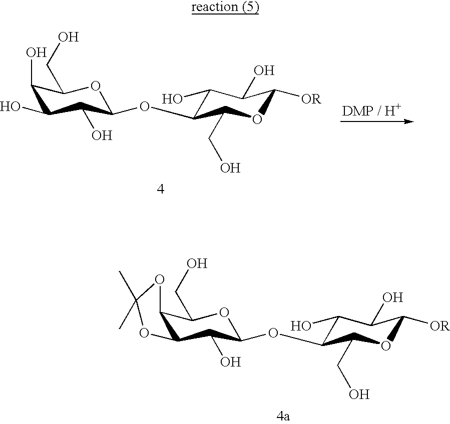

where R is as defined above.

The reaction is conducted under conditions to provide for 8-methoxycarbonyloctyl-3′,4′-O-isopropylidene-β-D-lactoside, compound 4a. Preferably, the reaction is conducted by reaction of compound 4 with 2,2-dimethoxypropane in the presence of an acidic catalyst, such as p-toluenesulfonic acid or (1S)-(+)-10-camphorsulfonic acid, to provide a pH of about 3 or less and at a temperature of from about 400 to about 100° C. and more preferably at about 65° C.

The reaction mixture is then poured into methanol, followed by neutralization. The resulting product is recovered by conventional methods including stripping of the solvent, chromatography, crystallization and the like, or can be used directly in the next reaction procedure without purification and/or isolation.

Each of the hydroxyl groups on 8-methoxycarbonyloctyl-3′,4′-O-isopropylidene-β-D-lactoside, compound 4a, is then protected by reaction with an excess of benzoyl chloride in an inert diluent as shown in reaction (6):

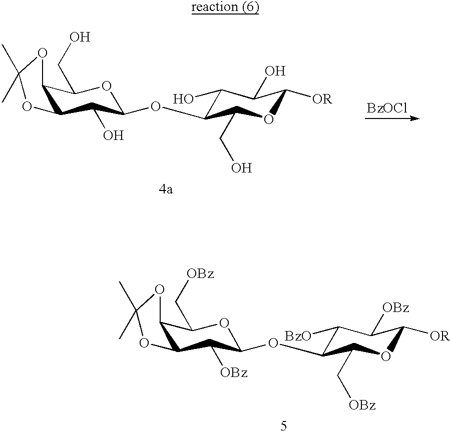

Reaction (6) is preferably conducted by combining compound 4a with an excess, i.e., at least 6 stoichiometric equivalents, of benzoyl chloride in a suitable inert diluent. Preferably, the amount of benzoyl chloride employed in the reaction is from at least 6 to about 10 equivalents and more preferably about 8 equivalents.

In one embodiment, an organic base such as triethylamine, DIEA and the like is added to the reaction mixture to scavenge the acid generated. In another embodiment, an inert diluent containing a basic group is employed. In this case, pyridine is employed as the preferred diluent.

The reaction is conducted under conditions to provide for 8-methoxycarbonyloctyl-3′,4′-O-isopropylidene-2,2′,3,6,6′-O-pentabenzoyl-β-D-lactoside, compound 5. Preferably, the reaction is conducted at a temperature of from about 10° C. to about 50° C. and more preferably at about room temperature for a period of from about 12 to about 48 hours. After reaction completion, methanol is preferably added to the reaction mixture to destroy any unreacted benozyl chloride. The resulting product is recovered by conventional methods including stripping of the solvent, crystallization and the like, and the purified product is then taken to the next step.

In one preferred embodiment, reaction (5) and (6) are conducted in a single pot and the resulting product is crystallized, e.g., in methanol, to remove any impurities thereby providing for 8-methoxycarbonyloctyl-3′,4′-O-isopropylidene-2,2′,3,6,6′-O-pentabenzoyl-β-D-lactoside, compound 5.

As illustrated in reaction (7), the dimethylacetal substituent of lactoside 5 is removed by conventional hydrolysis to provide for 8-methoxycarbonyl-octyl-2,2′,3,6,6′-O-pentabenzoyl-β-D-lactoside, compound 6.

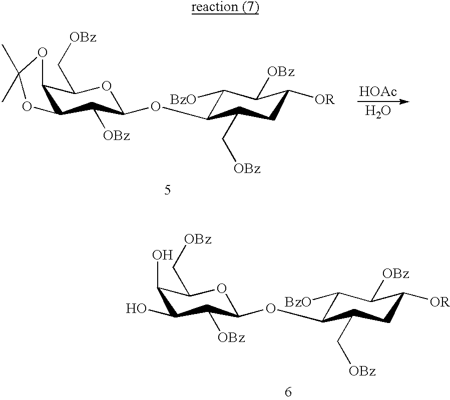

Preferred hydrolytic reaction conditions include the use of 80% acetic acid/water to effect hydrolysis. Such conditions are preferred because these conditions are sufficient to remove the acetal group but are not strong enough to remove a methoxy group from a methoxy carbonyl functionality on the aglycon [e.g., —(CH₂)₈COOCH₃_{]}. The reaction is preferably conducted at a temperature of from about 50° to 100° C. and more preferably at about 65° C. until the reaction is complete as evidenced by thin layer chromatography which is typically from about 12 to 48 hours. The resulting product can then be recovered by conventional methods including stripping of the solvent, chromatography, crystallization and the like, or can be used directly in the next reaction procedure without purification and/or isolation. 8-Methoxycarbonyl-octyl-2,2′,3,6,6′-O-pentabenzoyl-β-D-lactoside, compound 6, is then converted to the 4′-acetyl derivative, compound 7, by contacting this compound with triethylorthoacetate (TEOA) and an acid catalyst in an inert diluent (including, by way of example, dimethylformamide, acetonitrile, tetrahydrofuran, and the like) under conditions which provide for this compound as shown in reaction (8) below:

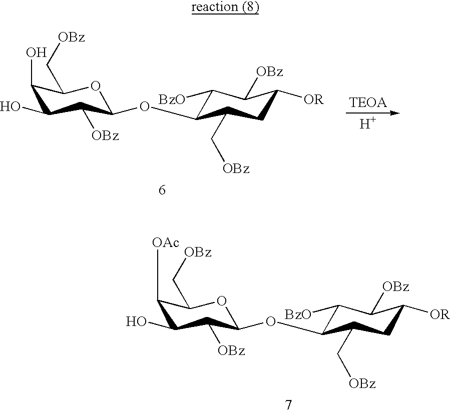

Reaction (8) results in protection of the 4′-hydroxy group by the removable acetyl blocking group thereby leaving only the 3′-hydroxy group available for linkage.

Preferably, reaction (8) employs from about 1 to about 5 equivalents and more preferably about 2 equivalents of triethylorthoacetate per lactoside 6. The acid catalyst employed is preferably p-toluene sulfonic acid or camphorsulfonic acid and the reaction is preferably conducted at a temperature of from about 0° to 40° C. and more preferably from about 15° to 25° C. until the reaction is complete as evidenced by thin layer chromatography which is typically from about 0.1 to 5 hours and preferably within about 1 hour.

Once this step is complete, the 4′-acetyl group is then generated by addition of an aqueous solution of hydrochloric acid to the reaction solution. Again, the reaction is preferably conducted at a temperature of from about 0° to 400C and more preferably from about 15° to 25° C. until the reaction is complete as evidenced by thin layer chromatography which is typically from about 0.1 to 5 hours and preferably within about 1 hour. The resulting 8-methoxycarbonyloctyl-4′-O-acetyl-2,2′,3,6,6′-O-pentabenzoyl-β-D-lactoside, compound 7, can then be isolated by conventional methods including stripping of the solvent, chromatography, crystallization and the like, or can be used directly in the next reaction procedure without purification and/or isolation.

Linkage of the terminal galactose group to compound 7 is accomplished by contacting compound 7 with tetra-O-benzyl-β-thiobenzyl galactoside, compound 8, in the presence of cupric bromide under conditions which provide for 8-methoxycarbonyloctyl-(2″,3″,4″,6″-tetra-O-benzyl-α-D-galactopyranosyl)(1→3)-(4′-O-acetyl-2′,6′-di-O-benzoyl-β-D-galactopyranosyl)-(1→4)-O-2,3, 6-tri-O-benzoyl-β-D-glucopyranoside, compound 9, as shown in reaction (9) below:

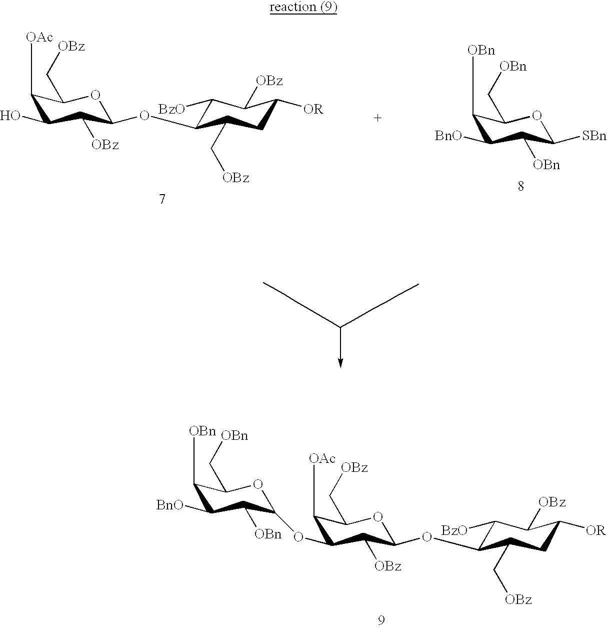

The 3′ hydroxy group of compound 7 and the benzyl protecting groups of compound 8 direct the reaction to formation of the α(1→3) linkage between the two galactose units.

Preferably, reaction (9) employs from about 1 to about 5 equivalents of the compound 8 per compound 7 and more preferably about 1.5 equivalents. Additionally, in a preferred embodiment the reaction employs from about 4 to about 6 equivalents of cupric bromide per equivalent of disaccharide 7 and about 8 to 12 equivalents of dimethylformamide. The reaction is conducted at a temperature of from about 0° to 60° C. and preferably at room temperature. The reaction is continued until complete as evidenced by thin layer chromatography which is typically from about 2 to 72 hours and preferably from about 16 to 48 hours. Suitable inert diluents include, by way of example, chloroform, methylene chloride, and the like. The resulting product can then be recovered by conventional methods including stripping of the solvent, chromatography, crystallization and the like, or can be used directly in the next reaction procedure without purification and/or isolation.

After formation of trisaccharide 9, the blocking groups are then removed by conventional methods as shown in FIG. 2 to provide for the desired αGal(1→3)βGal(1→4)Glc-OR compounds including 8-methoxycarbonyloctyl α-D-galactopyranosyl-(1→3)-O-β-D-galactopyranosyl-(1→4)-O-β-D-glucopyranoside (compound 11). The resulting product can be recovered by conventional methods, including crystallization.

As discussed above, the R aglycon preferably contains a functional group or can be derivatized to contain such a group which can provide for covalent linkage to a solid support. For example, as illustrated by Pinto, et al.¹² and Lemieux, et al.¹¹, the carboxymethyl (—COOCH₃) group of a —(CH₂)₈COOCH₃ aglycon can be converted under conventional conditions to the acyl azide derivative and then covalently linked to a solid support via an amide group. As discussed above, other aglycons which can be used to effect covalent linkage to a solid support can be found in Ekberg, et al.³, Dahmen, et al.⁴, Rana, et al.⁵, Amvam-Zollo, et al.⁶, Paulsen, et al.⁷, Chermnyak, et al.⁸, Fernadez-Santana, et al.⁹ and Lee, et al.¹⁰

Utility

The processes of this invention provide for the synthesis of compounds capable of binding toxin A and, accordingly, are useful in the treatment of antibiotic associated diarrhea mediated by, for example, Clostridium difficile which expresses toxin A.¹ When so employed, the compound, either by itself or attached to a pharmaceutically acceptable solid support, can be administered as a pharmaceutical composition to a patient suffering from antibiotic associated diarrhea. Oral administration of the compound coupled to a pharmaceutically acceptable solid support is preferred whereas, when the compound is not attached to a solid support, administration rectally is the preferred route.

These compounds can also be used in diagnostic assays for determining the presence of toxin A in a biological sample. For example, these compounds, appropriately labeled, can be used in a competitive assay to determine the amount and/or presence of toxin A in a compound. Suitable detectable labels include, by way of example, radiolabels, fluorescent labels, magnetic labels, enzymes, and the like. Attachment of a detectable label to these compounds is achieved via conventional methods well known in the art. Alternatively, the trisaccharide, containing an appropriate aglycon, can be attached to a solid support in the manner described above to provide a means to remove toxin A from a sample. After contact, the solid support is freed from the biological sample by conventional means such as washing, centrifugation, etc. Additionally, such solid supports can be used in a conventional ELISA assay to detect for the presence of toxin A in a biological sample such as a stool sample.

### EXAMPLES

The following examples are set forth to illustrate the claimed invention and are not to be construed as a limitation thereof.

Unless otherwise stated, all temperatures are in degrees Celsius. Also, in these examples, unless otherwise defined below, the abbreviations employed have their generally accepted meaning:

| | | |
|---|---|---|
| Å | = | Angstroms |
| Ac | = | acetyl |
| AgOTf | = | silver trifluoromethane sulfonate |
| Bn | = | benzyl |
| Bz | = | benzoyl |
| BzCl | = | benzoyl chloride |
| DCM | = | dichloromethane |
| DIEA | = | diisopropylethyl amine |
| DMP | = | dimethoxypropane |
| DMF | = | dimethylformamide |
| g | = | gram |
| IPA | = | isopropyl alcohol |
| kg | = | kilogram |
| L | = | liter |
| Pyr | = | pyridine |
| TLC | = | thin layer chromatography |
| μ | = | micron |

The synthetic scheme employed in the following examples is illustrated in FIGS. 1 and 2.

### Example 1

### Synthesis of 1,2,3,6,2′,3′,4′,6′-Octa-O-Benzoyl-β-Lactoside (Compound 2)

To a 20 L reaction vessel equipped with a mechanical stirrer is charged with 400 gm of commercially available lactose, compound 1 (1.10 mol, available from Aldrich) and pyridine (4.5 kg). While stirring the solution, 1.7 Kg of benzoyl chloride (12.3 mol, 12 equivalents) is added to the reaction vessel in 10±5 minutes. The reaction vessel is then heated to 65° C. and the reaction is stirred at this temperature for 24 hours. The progress of the reaction is monitored by TLC, using 85:15 of toluene:ethyl acetate to develop the silica gel plate.

Upon completion of the reaction as observed by TLC, excess benzoyl chloride is quenched by the addition of methanol (250 g). The reaction mixture is then concentrated under reduced pressure. The crude syrup is re-dissolved in 7 Kg DCM and washed with 7 Kg of water. The organic layer is then washed with 7 Kg of 5% hydrochloric acid solution and repeated until pH=1 is achieved. This is followed by washing with 7 Kg of 6% sodium bicarbonate solution. The organic layer is dried with sodium sulfate, filtered and taken directly to the next step as a stock solution.

### Example 2

### Synthesis of 2,3,6,2′,3′,4′,6′-Hepta-O-Benzoyl-a-D-Lactosyl Bromide (Compound 3)

To a 20 L reaction vessel equipped with a mechanical stirrer is charged with 10.5 Kg of DCM and 1400 g of 1,2,3,6,2′,3′,4′,6′-octa-O-benzoyl-β-D-lactoside (compound 2) prepared as per Example 1 above. While stirring the solution, 1110 g of 30% HBr/acetic acid (4.12 mol, 4.0 equivalents) is added. The reaction is stirred at room temperature for 16 hours. The progress of the reaction is determined by TLC using 3:2 of toluene:ethyl acetate to develop the silica gel plate.

Upon completion of the reaction, the organic layer is washed with 9 Kg of water followed by 2×9 Kg of 6% sodium bicarbonate solution. This washing step is repeated as necessary, until pH is neutral. The organic layer is removed under reduced pressure to give the title compound as syrup, which is extracted with hexanes. The residual hexane is evaporated and the compound is dried under high vacuum for 12 hours.

### Example 3

### Synthesis of 8-Methoxycarbonyloctyl-β-D-Lactoside (Compound 4)

PART #1

To a 30 L reaction vessel purged with nitrogen, equipped with a mechanical stirrer is charged 10 Kg of DCM followed by 1.5 Kg of 4 A° molecular sieves, 263 g of 8methoxycarbonyloctanol (1.40 mol, 1.2 equivalent), 147 g of 1,1′,3,3′-tetramethyl urea (1.26 mol, 1.1 equivalent) and 389 g of silver trifluromethanesulfonate (1.51 mol, 1.3 equivalent). To this reaction mixture is added 1300 g of 2,3,6,2′,3′,4′,6′-hepta-O-benzoyl-α-D-lactosyl bromide (Compound 3), dissolved in 2 Kg of DCM. The reaction mixture is stirred for 1 hour at ambient temperature and monitored by TLC using 85:15 of toluene:ethyl acetate as eluant.

Upon completion the reaction mixture is neutralized with 78 g of triethylamine to pH 6-7. The reaction mixture is filtered over Celite pad and rinsed with 3 Kg of DCM. The filtrate is then washed with 2×11 Kg of 5% ammonia solution, then with 11 Kg of water. The organic layer is neutralized with acetic acid (if the pH is >9) and then washed with 11 Kg of water. The organic layer is evaporated under reduced pressure to syrup. The syrup is then dried under high vacuum for a minimum of 12 hours.

PART#2

To a 20 L rotovap purged with nitrogen is charged 5.5 Kg of methanol and 8methoxycarbonyloctyl-2,3,6,2′,3′,4′,6′-O-heptabenzoyl-β-D-lactoside (Compound 3a) prepared as above. To this mixture, 300 g of 1N sodium methoxide is added to pH≧11. The reaction mixture is then heated to 45° C. and stirred until TLC shows reaction is complete.

The progress of the reaction is monitored by TLC using 65:35:8 of chloroform methanol water as eluant. Upon completion, the reaction mixture is neutralized by the addition of 26 g of glacial acetic acid to pH 5-7. The solvent is then removed by evaporation under reduced pressure. The crude product is extracted with 3.5 Kg of hexanes at ambient temperature for 2.0 hours. The solid is filtered and washed with 2×3.5 Kg of hexanes. The partially dried solid is dried by evaporation under reduced pressure until a free flowing solid is recovered. To the dried solid, 2.2 Kg of methanol and 4.75 Kg of ethyl acetate are added and the slurry stirred at 45° C. for 2 hours. The slurry is then stirred at ambient temperature for 12-24 hours followed by cooling to 0° C. for 2 hours. The solid is filtered, washed with ethyl acetate and hexanes, and dried under high vacuum to give the title compound.

### Example 4

### Synthesis of 8-Methoxycarbonyloctyl-3′,4′-O-Isopropylidene-2,3,6,2′,6′-O-Pentabenzoyl-β-D-Lactoside (Compound 5)

PART #1

To a 20 L reaction vessel is charged 400 g of β-D-Lactose grease, compound 4 (0.78 mol, 1 equivalent) in 3.4 Kg of 2,2′-dimethoxypropane. The reaction is heated to 65° C., followed by addition of 27 g of (1S)-(+)-10-camphorsulfonic acid to pH≦3. The reaction is complete once all starting material has been consumed as observed by TLC, using 4:1 of ethyl acetate: methanol as eluant to develop the silica gel plate.

The reaction mixture is cooled to ambient temperature and then added directly into 6.3 Kg of methanol and stirred for 10 minutes. This is followed by neutralization using 16 g of triethylamine. Once neutralized, the reaction mixture is analyzed by TLC using 4:1 of ethyl acetate: methanol. The reaction mixture is evaporated under reduced pressure to give a waxy solid, which is dried under high vacuum for a minimum of 12 hours. The dried solid is taken directly to the next step without any further purification.

PART#2

To a 20 L reaction vessel purged with nitrogen is charged compound 4a dissolved in 4.6 Kg of ACS grade pyridine. To the reaction mixture, 937 g of benzoyl chloride (6.6 mol, 8 equivalent) is added quickly while maintaining a reaction temperature below 50° C. The reaction is stirred at ambient temperature for 16-24 hours.

The reaction is monitored by TLC using 85:15 of toluene:ethyl acetate as eluant to develop the silica gel plate. Upon completion of the reaction, 286 g of methanol is added slowly to the rapidly stirred reaction mixture to consume the excess of benzoyl chloride.

The reaction mixture is evaporated under reduced pressure. The solid residue is dissolved in 8 Kg of DCM and washed with 6.6 Kg of water. The recovered organic layer is washed with 5.8 Kg of 5% hydrochloric acid (until pH=1), with 5.8 Kg of 6% sodium bicarbonate, and finally with 5.8 Kg of water. The organic layer is evaporated to dryness followed by 2×3 Kg hexane extractions to remove methyl benzoate. The recovered syrup is dissolved into 8 Kg of ACS grade methanol at 60±5° C. The crystallization is allowed to cool to room temperature and stir for 16-24 hours. The crystals are cooled to 0° C. and stirred for 3 hours. The crystals are filtered, and washed with methanol, then dried to give the title compound in 65% yield.

### Example 5

### Synthesis of 8-Methoxycarbonyloctyl-2,3,6,2′,6′-O-Pentabenzoyl-β-D-Lactoside (Compound 6)

To a 20 L reactor vessel equipped with a mechanical stirrer is charged with 560 g of 8-methoxycarbonyloctyl-3′,4′-O-isopropylidene-2,3,6,2′,6′-O-pentabenzoyl-β-D-lactoside, compound 5 and 5.2 Kg of 80% acetic acid. The reaction mixture is heated at 65° C. for 16-24 hours until complete.

The reaction is monitored by TLC using 85:15:5 of toluene:ethyl acetate:methanol as eluant to develop the silica gel plate. The reaction is cooled to ambient temperature, dissolved in 6.0 Kg of DCM and washed with 5.0 Kg of water. The aqueous layer is back extracted with 3.0 Kg of DCM. The combined organic layer is washed with 8.0 Kg of water, 8.0 Kg of 6% sodium bicarbonate, and finally with 8.0 Kg of water.

The organic layer is evaporated under reduced pressure and dried under high vacuum for 12 hours to give the title compound. The compound is used directly in the next synthetic step without any further purification.

### Example 6

### Synthesis of 8-Methoxycarbonyloctyl-4′-O-Acetyl-2,3,6,2′,6′-O-Penta benzoyl-β-D-Lactoside (Compound 7)

To a 20 L reactor vessel purged with nitrogen is charged 540 g of 8-methoxycarbonyloctyl-2,3,6,2′,6′-O-pentabenzoyl-β-D-lactoside, compound 6 (0.52 mol) dissolved in 4.8 Kg of tetrahydrofuran. To the reaction mixture 171 g of triethylorthoacetate (1.05 mol, 2.0 equivalent) and 13 g of (1S)-(+)-10-camphorsulfonic acid (0.05 mol, 0.1 equivalent) is added. The reaction mixture is stirred for 1 hour and monitored by TLC using 8515:5 of toluene:ethyl acetate:methanol as eluant to develop the silica gel plate.

Once this step is complete, 5.0 Kg of a 5% hydrochloric solution is added to the reaction mixture. The reaction is stirred for 1 hour and the progress monitored by TLC. Upon completion, the reaction mixture is diluted with 10 Kg of DCM and washed with 10 Kg of water, 8.0 Kg of 6% sodium bicarbonate and finally with 8.0 Kg of water.

The organic layer is evaporated under reduced pressure to give the title compound.

### Example 7

### Synthesis of 8-Methoxycarbonyloctyl (2″,3″,4″,6″-teta-O-benzyl-α-D-galactopyranosyl)-(1≧3)-O-(4′-O-acetyl-2′,6′-di-O-benzoyl-β-D-galactopyranosyl)(1→4)-O-2,3,6-tri-O-benzoyl-β-D-glucopyranoside (Compound 9)

To a 20 L reactor purged with nitrogen and equipped with a mechanical stirrer is charged 530 g of 4 A° molecular sieves, 530 g of 8-methoxycarbonyloctyl 4′-O-acetyl-2,3,6,2′,6′-penta-O-benzoyl-β-D-lactoside, compound 7 (0.51 mol, 1 equivalent), 550 g of copper II bromide (2.46 mol, 5 equivalent) and 385 g of anhydrous DMF (5.27 mol, 10 equivalent) in 7.0 Kg of DCM. The mixture is stirred for 30 minutes at ambient temperature. To the mixture is added 478 g of 2,3,4,6 tetra-O-benzyl-β-thiobenzyl galactoside, compound 8 (0.73 mol, purchased from Pfanstiehl). The reaction is allowed to proceed at ambient temperature for 16-48 hours.

The progress is monitored by TLC until starting material has been consumed. An 85:15 ratio of toluene:ethyl acetate is used as eluant. The reaction mixture is filtered over a Celite pad and the filter pad is washed with DCM. The filtrate is extracted with 2×8 Kg of 5% sulfuric acid solution and with 8.0 Kg of 6% sodium bicarbonate. The organic layer is separated and evaporated under reduced pressure. The crude product is taken directly to the next step without any further purification.

### Example 8

### Synthesis of 8-Methoxycarbonyloctyl-(2″,3″,4″,6″-tetra-O-benzyl-α-Dgalactopyranosyl)-(1→3)-O-β-D-galactopyranosyl-(1→4)-O-β-D-glucopyranoside (Compound 10)

To a 20 L reactor purged with nitrogen is added 6 Kg of methanol and 1100 g of 8-methoxycarbonyloctyl-(2″,3″,4″,6″-tetra-O-benzyl-α-D-galactopyranosyl)-(1→3)-(4′-O-acetyl-2′,6′-di-O-benzoyl-β-D-galactopyranosyl)-(1→4)-2,3,6-tri-O-benzoyl-β-D-glucopyranoside, compound 9 (0.70 mol).

A 1.0 N sodium methoxide solution (470 g) is added until the combined solution has a pH≧11. The resulting mixture is stirred for 8-12 hours at 45° C until the starting material is undetected by TLC using 95:5 of DCM:MeOH as eluant.

Upon completion of the reaction, the solution is neutralized by the addition of 47 g of glacial acetic acid to pH 5-7. The reaction mixture is concentrated under reduced pressure to a syrup. The syrup is dissolved in 6.0 Kg of ethyl acetate, and washed with 6.0 Kg of water. The organic layer is separated and evaporated under vacuum. The syrup is extracted with 2×3.6 Kg of hexanes for 30 minutes. The hexane fraction is decanted and the crude product is purified through a silica gel cartridge (5 kg) equilibrated with 99 : of DCM:MeOH. The product is eluted using a gradient of DCM:MeOH (99:1, 98:2, 97:3 v/v). The effluent of the column is collected in fractions, which are monitored by TLC. The appropriate fractions are pooled together and evaporated under vacuum to provide the title compound 10.

### Example 9

### Synthesis of 8-Methoxycarbonyloctyl-α-D-galactopyranosyl-(1→3)-O-β-D-galactopyranosyl-(1→4)-O-β-D-glucopyranoside (Compound 11)

To a 12 L three necked flask is added 400 g of 8-methoxycarbonyloctyl(2″,3″,4″,6″,-tetra-0-benzyl-α-D-galactopyranosyl-(1→3)-O-β-D-galactopyranosyl-(1→4)-O-β-D-glucopyranoside, compound 10 (0.38 mol) in 6.0 Kg of methanol. The reactor is purged with nitrogen for 15 minutes and then 120 g of 10% palladium/carbon is added. The mixture is stirred and purged with nitrogen for 15 minutes. The nitrogen flow is stopped and hydrogen is introduced into the reactor.

The suspension is stirred for 12-24 hours until complete consumption of starting material is observed by TLC using 65:35:8 of CHCl₃:MeOH:H₂O as eluant to develop the silica gel plate. The solution is filtered through an Alsop Filter pad followed by 0.22 μm filtration.

The filtrate is evaporated to a residue, which is then dried under vacuum. The residue is dissolved in EtOH:IPA (2:1 ratio, v/v) at 60° C. and the resulting solution is allowed to cool to ambient temperature overnight. The crystallized compound is filtered, and washed with IPA to give the title compound 11.

Although the foregoing invention has been described in some detail by way of example, it will be apparent that certain modifications may be practiced within the scope of the appended claims.

What is claimed is:

1. A process for preparing αGal(1→3)βGal(1→4)Glc- aglycons which process comprises:
(a) contacting β-R-lactoside represented by the formula: 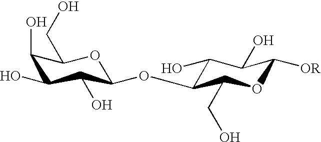
with at least a stoichiometric amount of a 2,2-dialkoxypropone under conditions to provide for β-R 3′, 4′-dialkylacetal lactoside of the formula: 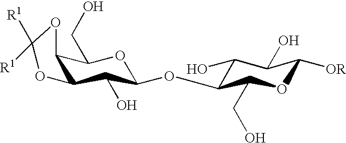
where R is an aglycon of at least I carbon atom and each R¹ is independently alkyl;
(b) acylating the compound produced in (a) above with at least 5 equivalents of an acyl halide under conditions to provide for β-R 3′,4′-dialkylacetal-2,2′,3,6,6′-penta-O-acyl-actoside of the formula: 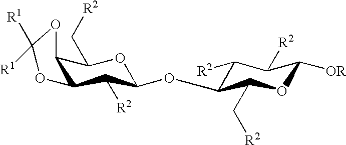
wherein R and R¹ are as defined above and R² is an acyl group;
(c) removing the dialkylacetal group from the compound produced (b) above to provide for the β-R 2,2′,3,6,6′-penta-O-acyl-lactoside of the formula: 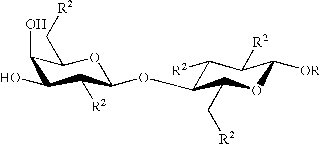
where R and R² are as defined above;
(d) acetylating the 4′-hydroxyl group of the the β-R 2,2′,3,6,6′-penta-O-acyl-lactoside by contacting the compound produced in (c) above with triethylorthoacetate under conditions to provide for the compound of the formula: 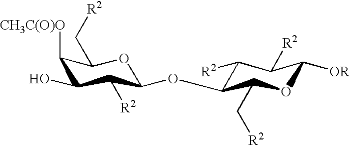
where R and R² are as defined above;
(e) contacting the compound produced in (d) above with P-thiobenzyl 2,3,4,6-tetra-O-benzyl-O-D-galactose under conditions to provide for a compound of the formula: 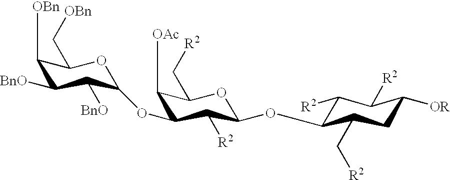
where R and R² are as defined above;
(f) removing each of the acetyl, benzyl and R² protecting groups in the compound produced in (e) above under conditions to provide for aglycon (α-D-galactopyranosyl)-(1→3)-O-(β-D-galactopyranosyl)-(1→4)-O-(β-D-glucopyranoside) which can also be referred to as αGal(1→3)βGal(1→4)Glc-OR and which is represented by the formula: 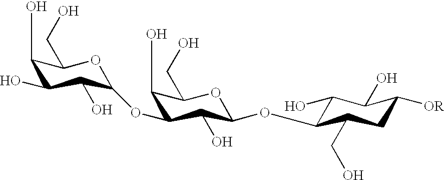
where R is as defined above.

2. The process according to claim 1 wherein the β-R lactoside represented by the formula: 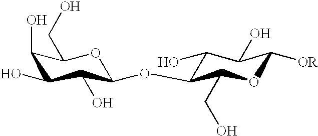
where R is as defined above, is prepared by the following process:
(i) contacting lactose of the formula: 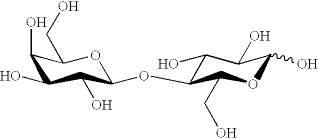
with at least 8 equivalents of benzoyl halide under conditions to provide for β-per-O-benzoyl-lactoside of the formula: 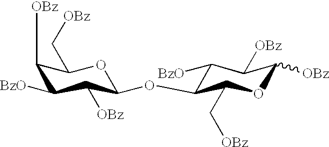
(ii) contacting β-per-O-benzoyl-lactoside prepared in (i) above with at least a stoichiometric amount of hydrogen bromide under conditions to form the 1-α-bromo derivative of the formula: 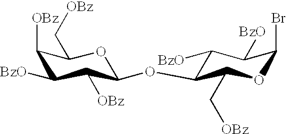
(iii) contacting the compound produced in (ii) above with a compound of the formula HOR under conditions to provide for β-OR-2,2′,3,3′,4′,6,6′-hepta-O-benzoyl lactoside of the formula; wherein R is an aglycon of at least 1 carbon atom; 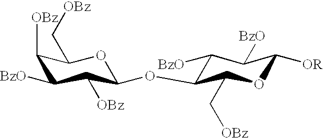
(iv) contacting the compound produced in (iii) above under conditions to debenzoylate said compound to provide for β-OR-lactoside.

3. The process according to claims 1 or 2 wherein R contains from 1 to 20 carbon atoms.

4. The process according to claim 3 wherein each R¹ is methyl.

5. The process according to claim 4 wherein each R² is acetyl.

6. A process for the synthesis of αGal(1→3)βGal(1→4)Glc-O(CH₂)₈—COOCH₃ which process comprises:
(a) contacting lactose of the formula: 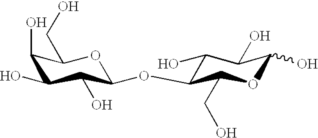
with at least 8 equivalents of benzoyl halide under conditions to provide for β-per-O-benzoyl-lactoside of the formula: 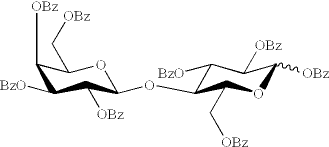
(b) contacting β-per-O-benzoyl-lactoside prepared in (i) above with at least a stoichiometric amount of hydrogen bromide under conditions to form the 1-α-bromo derivative of the formula: 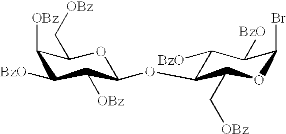
(c) contacting the compound produced in (b) above with a compound of the formula HOR under conditions to provide for β-OR-2,2′,3,3′,4′,6,6′-hepta-O-benzoyl lactoside of the formula; 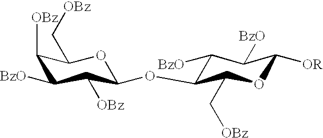
wherein R is —(CH₂)₈COOCH₃;
(d) contacting the compound produced in (c) above under conditions to debenzoylate said compound to provide for β-OR-lactoside of the formula: 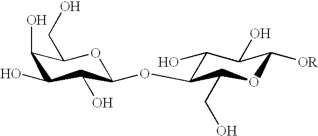
wherein R is as defined above;
(e) contacting the β-OR-lactoside produced in (d) above with at least a stoichiometric amount of a 2,2-dialkoxypropone under conditions to provide for β-R 3′,4′-dialkylacetal lactoside of the formula: 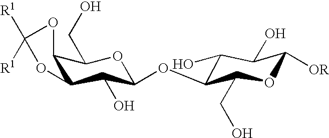
where each R¹ is independently alkyl;
(f) acylating the compound produced in (e) above with at least 5 equivalents of an acyl halide under conditions to provide for β-R 3′,4′-dialkylacetal-2,2′,3,6,6′-penta-O-acyl-lactoside of the formula: 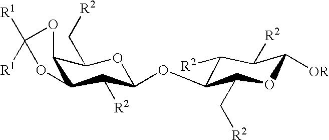
wherein R and R¹ are as defined above and R² is an acyl group;
(g) removing the dialkylacetal group from the compound produced (f) above to provide for the β-R 2,2′,3,6,6′-penta-O-acyl-lactoside of the formula: 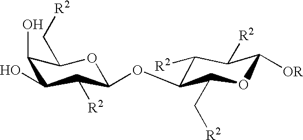
where R and R² are as defined above;
(h) acetylating the 4′-hydroxyl group of the the β-R 2,2′,3,6,6′-penta-O-acyl-lactoside by contacting the compound produced in (g) above with triethylorthoacetate under conditions to provide for the compound of the formula: 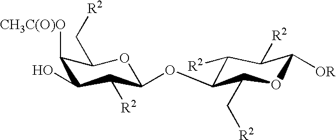
where R and R² are as defined above;
(i) contacting the compound produced in (h) above with β-thiobenzyl 2,3,4,6-tetra-O-benzyl-O-D-galactose under conditions to provide for a compound of the formula: 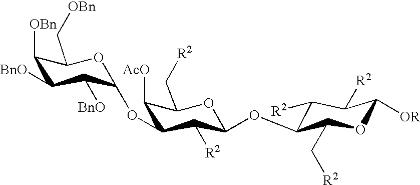
where R and R² are as defined above;
(j) removing each of the acetyl, benzyl and R² protecting groups in the compound produced in (e) above under conditions to provide for αGal(1→3)βGal(1→4)Glc-OR and which is represented by the formula: 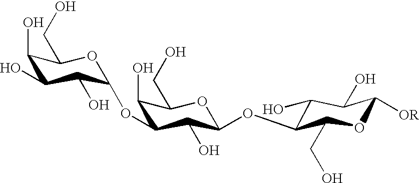
where R is as defined above.

7. The process according to claim 6 wherein each R¹ is methyl.

8. The process according to claim 7 wherein each R² is acetyl.
